(19) United States
(12) Patent Application Publication
(10) Pub. No.: US 2023/0045997 A1
(43) Pub. Date: Feb. 16, 2023

(54) BETA-GLUCAN FOR USE IN MODULATION OF AN IMMUNE RESPONSE IN A REMISSION

(71) Applicant: PLEURAN, S.R.O., Bratislava (SK)

(72) Inventors: Jan SPACEK, Praha (CZ); Eva ZAVADOVA, Praha (CZ); Bohuslav KONOPASEK, Praha (CZ); Jan GABRIZ, Bratislava (SK); Martin POLAK, Bratislava (SK)

(73) Assignee: PLEURAN, S.R.O., Bratislava (SK)

(21) Appl. No.: 17/759,660

(22) PCT Filed: Jan. 28, 2021
(86) PCT No.: PCT/IB2021/050666

§ 371 Date: Jul. 28, 2022

(30) Foreign Application Priority Data
Jan. 29, 2020 (EP) EP20154270.1
Jan. 29, 2020 (SK) SK PP 50004-2020

(51) Int. Cl. A61K 31/716; A61K 9/00; A61P 37/04

(52) U.S. Cl. CPC A61K 31/716; A61K 9/0053; A61P 37/04

(57) ABSTRACT

A use of beta-glucan in order to increase anti-tumor immunity in remission after the treatment of solid tumors, where after the long-term peroral usage the concentration of the CD8+ Lymphocytes, CD19+ lymphocytes increases and the level of IgG3, IgA, CD16+56+ increases. The clinical study has shown the efficiency of the preventive treatment during the immunosensitive cancer such as breast cancer. In the preferable arrangement beta-glucan is fungal β (1,3/1,6) glucan prepared from the oyster mushroom. During the sequential usage in the first phase a high dose of beta-glucan is used and in the second phase a low dose of beta-glucan is used. The high dose of beta-glucan is at least twice the low dose of beta-glucan. The administration is long-term and continuous without sequences where the dosage is completely omitted. The daily high dose of beta-glucan range from 600 mg to 800 mg.

### CROSS REFERENCE TO RELATED APPLICATION

This application is a national stage entry of PCT/IB2021/050666 filed Jan. 28, 2021, under the International Convention and claiming priority over European Patent Application No. EP20154270.1 filed Jan. 29, 2020 and Slovakia Patent Application No. SK PP 50004-2020 filed Jan. 29, 2020.

### FIELD OF TECHNOLOGY

The invention concerns the use of beta-glucans, mainly fungal β (1,3/1,6), in remission after primary oncological treatment, mainly after the treatment of the immunosensitive cancer such as colorectal cancer and breast cancer. The increase in anti-tumor cellular immunity has been reliably recorded during the peroral long-term use of fungal β (1,3/1,6) glucan, preferably in sequential dosage.

### PRIOR STATE OF THE ART

Anti-tumor cellular immunity is based on T lymphocyte activation and subsequent recognition of tumor antigens. Tumor-transformed cells originate in tissues and they are eliminated by T lymphocytes. The presence of CD8+ cytotoxic lymphocytes in the tumor microenvironment is considered to be a significant positive prognostic factor in patients with locally advanced colorectal cancer (Galon, Pages, et al. 2018).

Publication US2015064199 A1 discloses the method of use of neutral dissolved glucan and monoclonal antibodies for anti-tumor therapy. Neutrally dissolved β (1,3/1,6) glucan increases the tumoricidal activity of the innate immune system by binding the C3 complement to the CR3 protein receptor.

Publication US2001043914 A1 tackles the prevention and treatment of the tumors by use of slow-releasing microparticles containing IL-12 which are injected directly to the tumor.

Publication EP1651676 B1 discloses the method of implementation of substances to the cells by means of orally administered beta-glucan.

Publication US 2010/0166751 A1 discloses the use of beta-glucan for suppression and elimination of tumor cells in primary treatment. Beta-glucan pursuant to this publication is isolated from the cell walls of the yeasts. The use of beta-glucan in the primary treatment does not have convincing and verified results, yet.

Although breast cancer was not considered to be a so-called immunogenic tumour (for example, in comparison with malignant melanoma), it has been shown that the immune system also plays an important role in it. The state of antitumour cellular immunity (CD8+ cytotoxic effector lymphocytes) and the concentration of some immunomodulatory cytokines are gaining in clinical importance. Their monitoring and eventual modulation seem to be a promising way to find new biomarkers for immunotherapy and may be a way to improve its effectiveness.

Publications Taguchi T et al. “Life span prolongation effect of lentinan on patients with advanced or recurrent colorectal cancer” International Journal of Immunopharmacology, Elmsford, 1982, and Jinn-Chyi Wang et al. “Optimization for the production of water-soluble polysacharide from Pleurotus citrinopileatus in submerged culture and its antitumor effect” Applied Microbiology and Biotechnology, Springer 2005, disclose antitumor effects of the beta-glucan during primary treatment of the specific tumors. Publication WO0247612 hints towards this feature of beta-glucans in general.

Patent file WO 2008/057501 A2 discloses a lengthening of the remission of the cancer by means of beta-glucan enriched by O-acetyl groups. This publication does not disclose the timing of dosage and dosage regimen, and neither does it suggest that the dosage is determined by the expert during the determination of the course of therapy.

An increase of the anti-tumor cell immunity in the remission phase after primary treatment is desired, whereby the mean for increase of such anti-tumor immunity should not cause, even after long-term use, the worsening of organ functions, mainly functioning of liver, kidneys, and so on.

### SUMMARY OF THE INVENTION

The abovementioned deficiencies are significantly remedied by a beta-glucan-based polysaccharide immunomodulator which is used for modulation of immunity response during remission after the treatment of solid tumors no earlier than 6 months after the primary oncological treatment ended. The essential feature of the invention is the use of beta-glucan in order to increase increase of the antitumor cell immunity in remission after primary treatment of solid tumors. The primary treatment can include surgery and/or radiotherapy and/or chemotherapy and/or hormonal therapy and/or targeted biological therapy. Effective use of the beta-glucan in remission is proved irrespective of the type of primary oncological treatment, whereby significant results were recorded in cases of immunosensitive cancer such as colorectal cancer and breast cancer. In cases of these widespread oncological diagnoses the proofs of the significance and interpretation of the tumor-infiltrating lymphocytes are, in general, accepted. These are malignant diseases where one can expect the overall beneficial contribution of the immunomodulation in sense of stimulation of the cellular immunity response.

### BRIEF DESCRIPTION OF DRAWINGS

Results and effects of the invention are further disclosed by means of FIGS. 1 to 14.

FIG. 1 is a graph Ts-lymfo showing a significant increase of the concentration of the absolute number of CD8+ (cytotoxic T lymphocytes) during 12 months after the start of the clinical test;

FIG. 2 is a graph CD16+56+ showing a significant increase of the percentual share of CD16+56+ (NK calls) in 15 months after the start of the clinical test;

FIG. 3 is a graph CD19+ showing the comparison of the percentual share of B lymphocytes at the beginning of the use (0 months) and for period of 3 months;

FIG. 4 shows the sequential dosage regimen;

FIG. 5 is a graph IgG3 showing a comparison of the concentration of IgG3 at the start of the use (0 months) with the concentration of IgG3 after 3 months of use of β (1,3/1,6) glucan at dosage 700 mg/day;

FIG. 6 is a graph IgA showing a comparison of the concentration of IgA after the conclusion of sequence with high dose 700 mg/day in 9th month;

FIG. 7 is a graph showing a comparison of the percentage of CD19+ (B lymphocytes) at the beginning of use (0 months) and the percentage of CD19+ (B lymphocytes) after 3 months of beta-glucan use at a dose of 700 mg/day;

FIG. 8 a graph showing the results after 3 months from the start of the study in the control group;

FIG. 9 shows a graph showing a comparison of the IgA concentration after discontinuation of beta-glucan use at a concentration of 700 mg/day (9 months) with the concentration of IgA after 3 months (12 months) of beta-glucan use at a dose of 200 mg/day;

FIG. 10 is a graph showing a comparison of IgA concentration between 9 months and 12 months;

FIG. 11 is a graph showing a comparison of the percentage of CD3+ lymphocytes after discontinuation of the use of beta-glucan at a concentration of 200 mg/day (12 months) with the percentage of CD3+ lymphocytes after 3 months (15 months) of beta-glucan use at a dose of 200 mg/day;

FIG. 12 a graph showing a comparison of the percentage of CD3+ lymphocytes between 12 months and 15 months;

FIG. 13 is a graph showing how after 12 months from the start of the study, there was a significant increase in the concentration of absolute CD8+ (cytotoxic T lymphocytes) counts for the 700-100-700-200/day dosing regimen in the beta-glucan group (p=0.0147); and

FIG. 14 is a graph showing how after 12 months from the start of the study in the control group, there was a significant decrease in the concentration of absolute CD8+ (cytotoxic T lymphocytes) (p=0.04282).

### DESCRIPTION OF THE INVENTION

Significant preventive and therapeutic effects during the continuous long-term use of beta-glucan in remission during the treatment of solid tumors, mainly breast cancer, follow from the clinical study; the concentration of CD8+ lymphocytes increased alongside subsequent increase of CD19+ lymphocytes.

The term “increase of anti-tumor immunity” in this text denotes any change of the physiological rations of the cells in such a way that it increases the ability to eliminate the tumor-transformed cells. This increase of anti-tumor immunity is usually accompanied by increased concentration of CD8+ lymphocytes, CD19+ lymphocytes, increase of IgG3, IgA, CD16+56+, whereby the demarcation of boundary where the process is preventive and where is it therapeutic is not important.

The mechanism of β-glucan's action is mediated through several receptors, especially the dectin-1 receptor, toll-like receptors (TLR 2, 4, and 6), complement receptor 3 (CR3), scavenger receptor, and lactosylceramide.

The most important is the dectin-1 receptor, which is highly expressed in many immunocompetent cells, such as dendritic cells, neutrophils, eosinophils, macrophages, monocytes, and several T lymphocytes.

Predictors of response across all breast cancer subtypes include increased CD8+ cytotoxic T cells, CD4+ follicular T helper cells, and CD20+ B cells. The presence of TILs may be predictive of significance even for neoadjuvant and adjuvant therapy. The increased number of TILs correlates with the likelihood of achieving pathological complete remission in patients treated with neoadjuvant anthracycline chemotherapy with taxanes in triple-negative cancers and HER2+ cancers, especially with carboplatin therapy. Elevated TILs in ER-negative breast cancers predict predictive complete remission with anthracycline-based neoadjuvant chemotherapy, but not with CMF (cyclophosphamide, methotrexate, 5-fluorouracil) regimens. The presence of TILs may also predict the risk of recurrence after adjuvant treatment with trastuzumab in HER2+ cancers. In patients with HER2-positive tumours and present TILs, pathological complete remission was achieved in 40% of cases compared to 11% in HER2-negative tumours.

An active antitumour cellular immunity usually correlates with improved chances of survival and better prognosis of the patient. In breast cancer, some studies have reported that cytotoxic lymphocyte infiltration is associated with better survival. It has been shown that CD8+ lymphocyte infiltration is an independent favourable prognostic indicator in basal-like breast cancer.

CD8+ T cell-mediated type 1 immune responses can enhance the accumulation of distinct endogenous CD8+ and CD4+ T cells and facilitate their antitumour function within the tumour microenvironment.

The TLR pathway involves numerous proteins, which are known as potential oncogenes/tumour suppressors like IKKε and MITA. Modulation of these TLRs in tumour cells might help to secrete specific cytokines having antitumouric effects such as IFNs and TNFα. These cytokines might act in an autocrine or paracrine manner to stimulate nearby tumour cells, thus inhibiting the tumour growth.

Breast cancers are comprised of heterogeneous subtypes of various prognoses. Hormonal therapy is the basic treatment for patients with hormonal dependent breast cancer. Increased expression of hormonal receptors is an important condition of the effectiveness of hormonal therapy. Despite a rise in the use of selective aromatase inhibitors, antiestrogen therapy is still very important. Oestrogen's modes of actions are transmitted into the cell through nuclear receptors (estrogen receptors (ER)/progesterone receptors (PR)). After activation, those receptors serve (together with other regulatory molecules) as transcription regulators. Increased expression of hormonal receptors is an important condition in the effectiveness of hormonal therapy. Antioestrogens are the base of hormonal treatment in patients with hormone-dependent breast cancer, and those patients with the metastatic disease receive hormonal therapy in the neoadjuvant as well as in the adjuvant indication. Although more than ⅔ of treated patients respond to hormonal therapy, the effectiveness of hormonal treatment is mostly time-limited as most patients develop a resistance to this treatment. Developing resistance in patients with hormonal dependent carcinomas is a complex process, which interferes with intracellular signal transduction on the molecular level. Endocrine therapy is widely used for oestrogen-receptor-positive breast cancer. However, many of these patients experience a recurrence despite endocrine therapy by an incompletely understood mechanism.

The proliferation of many breast cancer cells is under the control of sex steroids oestrogen and progesterone, and such proliferation allows for an expansion of tumour tissue which requires new blood vessels for nourishment. Oestrogens influence angiogenetic processes resulting into metastasis particularly through vascular endothelial growth factor (VEGF), natural and synthetic progestins induce VEGF in breast cancer cells through the progesterone receptor. VEGF is a key mediator of angiogenesis. VEGF-targeting therapies have shown significant benefits and have been successfully integrated in the routine clinical practice for other types of cancer, such as metastatic colorectal cancer. An increasing amount of data is evolving from preclinical models, suggesting that ovarian steroids cause cyclical changes that have an impact on angiogenesis, and that oestrogen modulates angiogenesis, in part, through the effects on VEGF. Progesterone mechanisms involved in the progesterone effects on genes are implicated in the control of cell cycle, proliferation, angiogenesis and metastasis, such as epidermal growth factor receptor (EGFR), whose promoters lack sequences, and VEGF, whose gene contains progesterone response elements in its promoter region. Therefore VEGF, as well as TGF beta, are suggested to act as predictive markers of the disease progression. VEGF is a multifunctional glycoprotein acting as a specific mitogen for endothelial cells and increasing vascular permeability as well. High VEGF levels were described in various human cancer types (breast cancer, endometrial cancer and ovarian cancer).

Contrary to the methods known in the prior art, the beta-glucan according to this invention is used for long term and perorally, which can be easily ensured if the patient cooperates. Clinical study has shown a preventive anti-cancer effect during the long-term use without undesired side effects during simultaneous decrease in the incidence of the common infections.

First significant changes have been recorded after 3 months in the humoral component of the immune system; these changes appeared as significant increase of B lymphocytes and immunoglobulin. First significant changes in the cellular immunity (NK cells and CD8+ lymphocytes) took place only after longer period of use of beta-glucan. Immunomodulation by means of beta-glucan is therefore, pursuant to this invention, recommended in the adjuvant mode. Immunity response takes place with a longer lag after the initiation of therapy; significant improvement of the immunological parameters takes place 12 to 15 months after the beginning of the use, which is why beta-glucan is suitable for long-term administration.

The result of the invention is clear effect of the perorally used preparation on the support of the immune system of the individual in period of so-called complete remission. Significant adjustments of the physiological ratios of the cellular anti-tumor immunity during continuous use of the beta-glucan serve the purpose of restoration of the immune system and alleviation of secondary immunodeficiency in cancer patients, e.g. in patients with hormone-dependent breast cancer. Analogical results are expected, on the basis of verified mechanism of the effects, in case of the remission during the colorectal cancer, or after treatment of endometrial cancer and ovarian cancer.

Beta-glucan as a preparation according to this invention is in the preferably arrangement a fungal β (1,3/1,6) glucan. Such beta-glucan can be prepared from the oyster mushroom (Pleurotus ostreatus), when such beta-glucan is also called pleuran.

The subject matter of the protection is also a composition which includes beta-glucan according to the description, whereby it is adapted for the oral use, for example, in form of capsules. The composition can—aside from beta-glucan—also include vitamin C and, possibly, some bacterial cultures, oligosaccharides, adjuvants, conditioning agents such as preservatives, and so on.

Beta-glucan is preferably administered to patients which are in remission at least for 6 months—that is, it is administered after 6 months since the conclusion of the primary oncological treatment at earliest; preferably after 12 months since the conclusion of the primary oncological treatment at earliest.

The dosage regimen (regime of dosing, dosage regime) of beta-glucan is also subject of protection, whereby this dosage regimen demonstrably leads to effective results. The essence of the sequential dosage regimen is the alteration of repeated phases. Beta-glucan is used continually, without a stage of zero dosage, in two subsequent and subsequently repeated phases, whereby during the first phase a high dose of beta-glucan is used and in the second phase a low dose of beta-glucan is used. These two phases are subsequently altered, therefore the names “first” and “second” are only used to distinguish them. High dose of beta-glucan is at least twice the low dose. High dose of beta-glucan can range from 600 mg to 800 mg, preferably 700 mg; low dose of beta-glucan can range from 50 mg to 300 mg, preferably 100 mg to 200 mg.

Pursuant to the results of clinical study it is preferable if the high dose has constant level during the use and the low dose regularly alters between values 100 mg and 200 mg. First and second phase can last for the same period of time, that is, 2 to 4 months, preferably 3 months.

Beta-glucan (polysaccharide beta-glucan-based immunomodulator) brings, during its long-term use, significant improvement of all crucial oncomarkers (tumor markers); it is manifested as significant preventive preparation, whereby no patient from the assessed group has fallen into relapse. The advantage of beta-glucan is an absence of the undesired side effects. On contrary, the administration of beta-glucan leads to positive side effects in the immunological picture of patients.

Our results suggest that immunomodulation and stimulation of CD8+ lymphocytes could refine the prediction of resistance and contribute to any targeted immune intervention therapy and targeted selection of cancer treatment. In the future, this finding could help restore the immune system of patients with solid tumours before immunotherapy. More studies are necessary in order to show whether those factors can be used for adjusting individual therapy in patients with hormonally dependent breast cancer.

Besides the significant decrease of CD8 T lymphocytes we observed in the control group also decrease of total CD3 (n.s). T lymphocytes with cytotoxic function (CD8) are forced and exhausted because of recurrent infections, probably also due to IgG and IgA immunodeficiency. In cancer patients, immunity defence is decreased not only because of cancer disease, but also owing to repeated infections connected with IgG immunodeficiency. Immunosuppression and insufficient anticancer immune defence might also be due to an exhausted immune system caused by repeated bacterial and viral infections. Based on our results and experience, we assume that describing and investigating basic immunity parameters in cancer patients should be a part of oncology care. In the case of proven immunodeficiency, patients may benefit from immunomodulatory therapy according to the guidelines used in clinical immunology in the treatment of immunopathologic disorders.

Demonstration of a significant adjustment of the physiological conditions of cellular antitumour immunity with continuous administration of betaglucan (insoluble β-1,3/1,6-D-glucan isolated from Pleurotus ostreatus) immunoglucan could in the future serve to restore the immune system and alleviate secondary immunodeficiency in cancer patients, e.g., (in patients with hormone-dependent breast cancer). We observed the first significant changes after 3 months in the humoral component of the immune system (significant increase in B lymphocytes and immunoglobulins.) Significant changes in cellular immunity (NK cells and CD8+ T lymphocytes) occurred only after prolonged use of betaglucan. No changes in liver, kidney or mineral biochemistry parameters were observed. Immunomodulation with betaglucan pleuran based an immunoglucan preparation can therefore be recommended in an adjuvant regimen. It shows that the immune response occurs with a longer interval after the start of therapy, a significant improvement in immunological parameters (12-15 months), therefore the betaglucan is suitable for long-term administration.

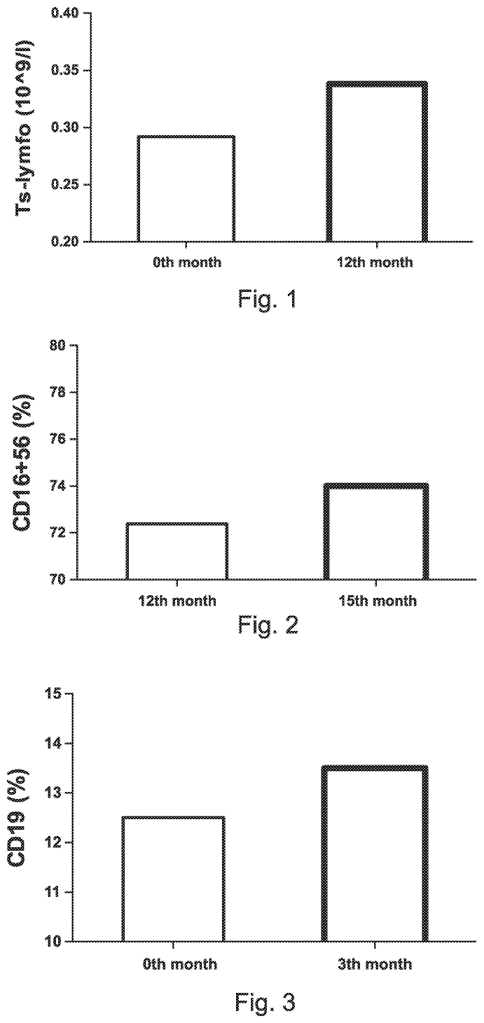

Graph Ts-lymfo in FIG. 1 shows a significant increase of the concentration of the absolute number of CD8+ (cytotoxic T lymphocytes) during 12 months after the start of the clinical test in the regime of sequential dosage with repeated 700 mg-100 mg-700 mg-200 mg/day phases in case of group of patients using β (1,3/1,6) glucan (p=0.0147). The value for the 12th month corresponds to end of 700 mg-100 mg-700 mg-200 mg/day dosage.

Graph CD16+56+ in FIG. 2 shows a significant increase of the percentual share of CD16+56+ (NK calls) in 15 months after the start of the clinical test in the regime of sequential dosage with repeated 700 mg-100 mg-700 mg-200 mg/day phases in case of group of patients using β (1,3/1,6) glucan (p=0.0313). The value for the 15th month corresponds to state at the end of 700 mg/day dose.

Graph CD19+ in FIG. 3 depicts the comparison of the percentual share of B lymphocytes at the beginning of the use (0 months) and for period of 3 months at dosage 700 mg/day. Significant increase of the percentual share of CD19+ took place after 3 months of usage of daily dose of 700 mg/day (p=0.0246). The value displayed for the 3rd month corresponds to state at the end of 700 mg/day dose.

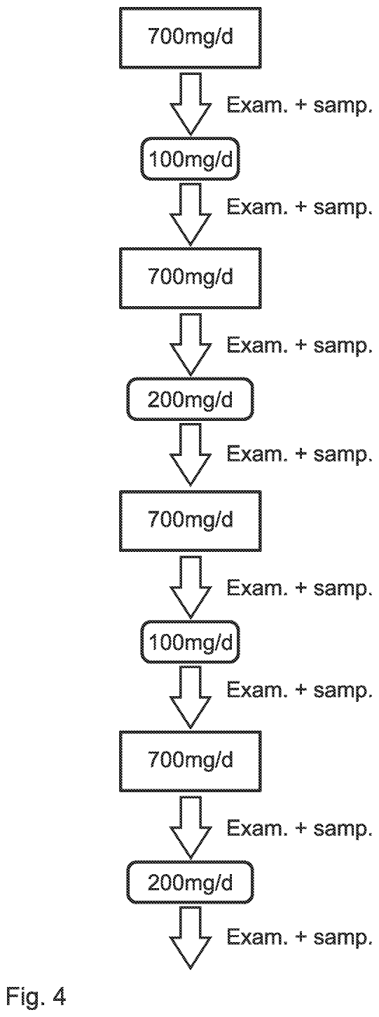

FIG. 4 depicts the sequential dosage regimen. The shortcut “Exam.” refers to patient examination; the shortcut “samp.” refers to sampling.

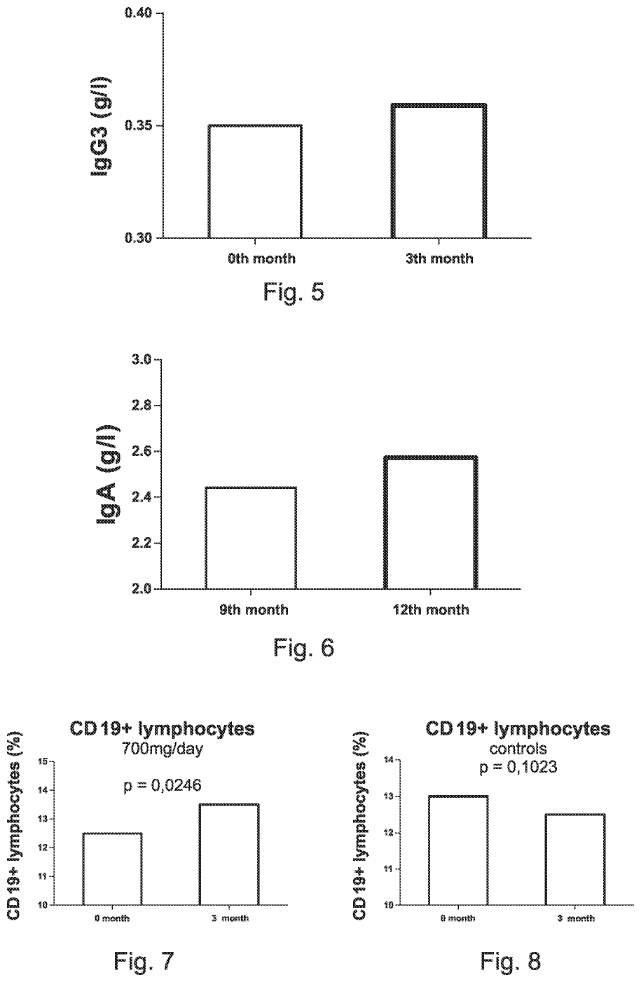

Graph IgG3 in FIG. 5 is a comparison of the concentration of IgG3 at the start of the use (0 months) with the concentration of IgG3 after 3 months of use of β (1,3/1,6) glucan at dosage 700 mg/day. Graph shows a significant increase in the concentration of IgG3 after 3 months of use (p=0.0010). The value displayed for 3rd month corresponds to state at the end of 700 mg/day dose.

Graph IgA in FIG. 6 is a comparison of the concentration of IgA after the conclusion of sequence with high dose 700 mg/day in 9th month and subsequently after three further months of use with low dose of β (1,3/1,6) glucan at 200 mg/day. The graph shows significant increase of the IgA concentration after 3 months of use, in total for 12 months since the start of the study (p=0.0255). The value displayed for 12th month corresponds to state at the end of 200 mg/day dose.

FIG. 7 depicts a comparison of the percentage of CD19+ (B lymphocytes) at the beginning of use (0 months) and the percentage of CD19+ (B lymphocytes) after 3 months of beta-glucan use at a dose of 700 mg/day. There was a significant increase in the percentage of CD19+ after 3 months of using beta-glucan at a dose of 700 mg/day (3 months from the start of the study) (p=0.0246).

FIG. 8 shows how after 3 months from the start of the study in the control group, there was a significant decrease in the concentration of absolute CD19+ (B lymphocytes) (p=0.1023).

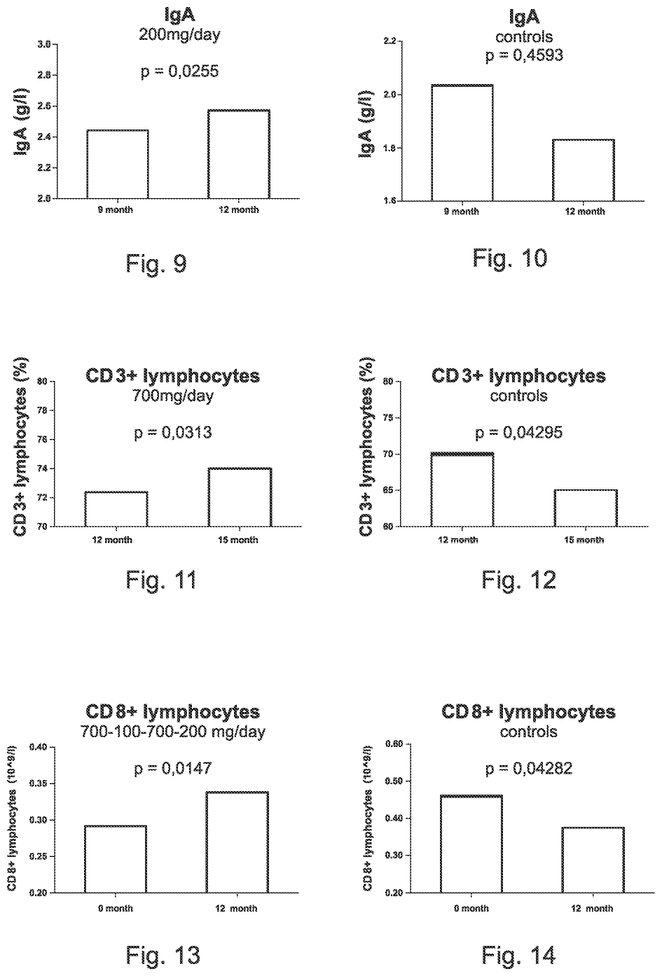

FIG. 9 depicts a comparison of the IgA concentration after discontinuation of beta-glucan use at a concentration of 700 mg/day (9 months) with the concentration of IgA after 3 months (12 months) of beta-glucan use at a dose of 200 mg/day. There was a significant increase in IgA levels after 3 months of using beta-glucan at a dose of 200 mg/day (12 months from the start of the study) (p=0.0255).

FIG. 10 depicts a comparison of IgA concentration between 9 months and 12 months. There was a significant decrease in IgA levels after 3 months (12 months from the start of the study) (p=0.4593).

FIG. 11 depicts a comparison of the percentage of CD3+ lymphocytes after discontinuation of the use of beta-glucan at a concentration of 200 mg/day (12 months) with the percentage of CD3+ lymphocytes after 3 months (15 months) of beta-glucan use at a dose of 200 mg/day. There was a significant increase in the percentage of CD3+ for the 700-100-700-200-700/day dosing regimen in the beta-glucan group (p=0.0313).

FIG. 12 depicts a comparison of the percentage of CD3+ lymphocytes between 12 months and 15 months. There was a significant decrease in IgA levels after 3 months (12 months from the start of the study) (p=0.04295).

FIG. 13 shows how after 12 months from the start of the study, there was a significant increase in the concentration of absolute CD8+ (cytotoxic T lymphocytes) counts for the 700-100-700-200/day dosing regimen in the beta-glucan group (p=0.0147).

FIG. 14 shows how after 12 months from the start of the study in the control group, there was a significant decrease in the concentration of absolute CD8+ (cytotoxic T lymphocytes) (p=0.04282).

### Example of Realization

### Example 1

In a clinical study with the results pursuant to FIGS. 1 to 6 the purpose was to determine the way in which immune system of the patients with hormonal-dependent breast cancer reacts to the continual use of β (1,3/1,6) glucan in clinical and display remission.

Clinical study involved 60 patients who underwent standard oncological treatment for the indication of locally advanced hormonal-dependent breast cancer of I-II clinical stage. The primary treatment of the cancer in the group involved various methods: surgery, radiotherapy, chemotherapy, hormonal therapy, pursuant to the individual indication. Before their inclusion in the clinical study the patients were examined by the clinical immunologist in order to rule out immunopathology and allergic or autoimmune underlying diseases. The anti-tumor immunity (CD4+, CD8+, B cells) were examined by a method of flow cytometry. TGF beta and VEGF were measured by ELISA method.

The patients used β (1,3/1,6) glucan perorally in a sequential regime, whereby they altered phases with high doses with phases with low doses, whereby the use was continuous without the zero dose phases. Beta-glucan (named “Imunoglukan”, which is the invention applicant's trademark) was in form of capsules which also included calcium L-ascorbate, magnesium stearate and benzoic acid.

Significant increase of B lymphocytes (CD19+) took place after three months of use of β (1,3/1,6) glucan with dosage 700 mg/day (p=0.0246). After three months of use a significant increase of level of IgG3 (p=0.0387) was recorded, too.

Significant increase of level of IgA took place after 12 months since the start of the study (p=0.0255).

Significant increase of CD16+56+ level took place after three months after repeated increase of the doses from 200 mg/day to 700 mg/day (15 months since the start of the study) (p=0.0469).

After 15 months since the start of the study a significant increase of concentration of CD8+ (cytotoxic T lymphocytes) at dosage regimen 700 mg-100 mg-700 mg-200 mg/day took place (p=0.0490).

During the long-term administration of β (1,3/1,6) glucan there were no changes in liver tests, renal function and autoimmune parameters. Clinically, Imunoglukan was very well tolerated by the patients and a decrease in incidence of infections (URT, uroinfections, gynecological infections, herpes) took place.

No patient from the observed group in the remission entered a relapse. The results of the clinical study pursuant to FIGS. 1 to 6 also show the increase of the effects during sequential dosage while the continuous use is ensured.

### Example 2

The aim of the study according to the FIGS. 7 to 14 was to seek new predictive markers of therapy response in breast cancer patients who were resistant to hormone therapy in particular immunity response by monitoring TGF beta and VEGF plasma levels as well as the cellular immunity response (CD8+, CD4+).

The open-label, controlled clinical study research project included 195 patients who underwent standard oncological treatment for the indication of locally advanced hormone-dependent breast cancer I-II clinical stage. The histological type and grade, the degree of expression of ER and PR, HER2, and the proliferative marker were established. Patients were treated according to standard therapy protocols and indications, in standard doses. The 49 women with hormonal dependent breast cancer, who used imunoglucan betaglucan pleuran based supplement (Neomun®—applicants trademark), were histologically confirmed as having invasive breast cancer and had a median age of 52 years. Of these women, 34 were postmenopausal (69%), and 15 premenopausal (31%); 49 women underwent surgery. Ablation with exenteration of axilla was done in 25 cases, partial resection with exenteration of axilla in 24 cases. From the histological point of view it was mainly invasive ductal carcinoma, namely in 37 cases. Lobular cancer was diagnosed in 8 cases and other histological types in 4 cases. All tumours were hormonal dependent; ER and PR were present in all of them. In 28 cases, ER was positive for more than 50% of the cells; in 6 cases, between 5 and 50% and in 15 cases under 5%; in 22 cases. PR was positive for more than 50% of the cells; in 10 cases, between 5 and 50% and in 17 cases under 5%; in all cases HER2 was negative. Patients in this study had similar treatment cycles.

All patients were further examined by a clinical immunologist to exclude immunopathology and diseases of the allergic or autoimmune basis. TGF beta and VEGF were measured by ELISA. Antitumour, cellular immunity (CD4+, CD8+, B cells) was examined by flow cytometry. A sequencing scheme was chosen for β-glucan administration.

escalation to 700 mg, the dose was again reduced to 200 mg daily. The aim was to find the optimal dosage for inducing favourable changes in immune parameters. The control group without β-glucan administration comprised 146 women with hormonal dependent breast cancer, with a median age of 53 years; 130 of the women were postmenopausal and 16 premenopausal.

Antitumour cellular immunity (CD4, CD8, NK cells, HLA-DR) was measured by flow cytometry. (Cytometr Navios, Software Navios, v.01 Beckman Coulter, monoclonal antibodies: CD4-APC-Alexa Fluor 750, clone 13B8.2, IgG1, cat. no. A94682 Beckman Coulter, CD8-Phycoerythrin-Cyanine7, clone SFCI21Thy2D3, IgG1, cat. no. 737661 Beckman Coulter, CD16-Phycoerythrin, clone 3G8, IgG1, cat. no. A07766 Beckman Coulter, CD3/HLA-DR-Fluorescein isothiocyanate/Phycoerythrin, clone UCHT1, IgG1/IgG1, cat. no. A07737 Beckman Coulter, Isotypic Control-Fluorescein isothiocyanate/Phycoerythrin, clone 679.1Mc7, IgG1/IgG1, cat. no. A07794 Beckman Coulter, CD45/CD56/CD19/CD3-Fluorescein isothiocyanate/Phycoerythrin/Phycoerythrin-Texas Red-X/Phycoerythrin-Cyanine5, clone B3821F4A/N901NKH1/J3-119/UCHT1, IgG2b/IgG1/IgG1/1gG1, cat. no. 6607073 Beckman Coulter). Determination of immunoglobulins was done by Beckman Coulter Image 800 Immunochemistry System (Nelphelometry). For statistical analysis, the Prism 4 (Graph Pad Software Inc.) was used. The relationships between variables were obtained using Spearman's nonparametric test, and differences between variables were determined by employing the Mann-Whitney U-test. A statistical comparison of intra- and inter-individual variation was carried out by the use of one-way analysis of variance (ANOVA).

There was a significant percentage increase in B cells (CD19+) after three months of using betaglucan pleuran at a dose of 700 mg/day (p=0.0246). There was a significant increase in IgG3 levels after 3 months of betaglucan pleuran use (p=0.0010). There was a significant increase in IgA levels 12 months after the start of the study (p=0.0255). There was a significant increase in CD3+ three months after re-increasing the dose from 200 mg/day to 700 mg/day (15 months from the start of the study) (p=0.0313). At 15 months from the start of the study, there was a significant increase in CD8+ (cytotoxic T cell) levels at the 700-100-700-200/day dosing schedule (p=0.0147) in the betaglucan pleuran group. There were no changes in liver function tests, renal function and autoimmune parameters during long-term administration of betaglucan. Clinically, betaglucan was very well tolerated by patients, and the incidence of infections (HCD, urinary tract infections, gynaecological infections, cold sores) was reduced.

This study aimed to examine the immunomodulatoryion potential of betaglucan (insoluble β-1,3/1,6-D-glucan isolated from Pleurotus ostreatus) in patients with hormonal-dependent breast cancer. In the times, when prognostic significance of immune response (CD8+) in breast cancer patients resistant to hormone therapy is proven.

Our hypothesis was that CD8+ lymphocyte population and the immune response is decreased in endocrine dependent breast cancer compared to the patients who were given betaglucan regularly.

In patients, who were enrolled in the control group, we found mainly defects of cellular antitumour immunity—lower expression of CD8+ (the cytotoxic T lymphocytes). Decreased humoral component of immunity, including the clinical manifestations of immunodeficiency, was found in many of these patients. Accumulating evidence indicates that malignant cells exert a major control on their non-malignant neighbours. Thus, most cancer cells not only promote angiogenesis to support tumour growth beyond the size limit that would be dictated by a poorly vascularised microenvironmen but also activate metabolic circuitries whereby stromal cells are de facto revived to function as a feeder compartment, generating large amounts of energetic products such as lactate and ketone bodies.

### INDUSTRIAL APPLICABILITY

Industrial applicability of this invention is obvious. According to this invention it is possible to industrially and repeatedly produce a preparation, or composition, respectively, for increase of the anti-tumor immunity in remission after primary treatment of the solid tumors.

1. A composition comprising beta-glucan for treating an immune response after treatment of a solid tumor, wherein the beta-glucan is administered no earlier than 6 months after a primary oncological treatment ends.

2. The composition according to claim 1, wherein the composition is administered in a remission after a breast cancer treatment.

3. The composition according to claim 1, wherein the composition is administered in a remission after a colorectal cancer treatment.

4. The composition according to claim 1, wherein the composition is administered in a remission after an endometrial cancer treatment.

5. The composition according to claim 1, wherein the composition is administered in a remission after an ovarian cancer treatment.

6. The composition according to claim 1, wherein the composition is administered in a modulation of an immune response in a remission after a treatment of solid tumors no earlier than 12 months after the primary oncological treatment ends.

7. The composition according to claim 1, wherein the composition increases a concentration of CD8+ lymphocytes while simultaneously increasing a concentration of CD19+ in a remission.

8. The composition according to claim 1, wherein the beta-glucan is fungal beta-glucan.

9. The composition according to claim 1, wherein the beta-glucan is fungal β (1,3/1,6) glucan.

10. The composition according to the claim 8, wherein the fungal beta-glucan is prepared from an oyster mushroom (Pleurotus ostreatus).

11. The composition according to claim 8, wherein the composition is an oral administration composition.

12. The composition according to claim 1, wherein the composition is administered continuously in subsequent a first and a second phases, whereby during the first phase a first dose of beta-glucan is administered and the second phase a second dose of beta-glucan is administered, wherein the first dose of beta-glucan is at least twice of the second dose of beta-glucan and the second dose is non-zero.

13. The composition according to claim 12, wherein the first dose of beta-glucan ranges daily from 600 mg to 800 mg and the second dose of beta-glucan ranges daily from 50 mg to 300 mg.

14. The composition according to claim 12, wherein the first dose has a constant level during the use and the second dose regularly alternates between values 100 and 200 mg.

15. The composition according to claim 12, wherein the first phase and the second phase last 2 to 4 months.

16. The composition according to claim 12, wherein the first phase and the second phase have identical temporal duration.
